(19) United States
(12) Patent Application Publication
(10) Pub. No.: US 2025/0186778 A1
(43) Pub. Date: Jun. 12, 2025

(54) SYSTEMS AND METHODS FOR ADAPTIVE NEUROMODULATION BASED ON EVOKED RESPONSES

(71) Applicant: Boston Scientific Neuromodulation Corporation, Valencia, CA (US)

(72) Inventors: G. Karl Steinke, Valencia, CA (US); Max Winter, Grand Junction, CO (US)

(21) Appl. No.: 18/976,076

(22) Filed: Dec. 10, 2024

Related U.S. Application Data
(60) Provisional application No. 63/609,147, filed on Dec. 12, 2023.

(51) Int. Cl. A61N 1/36; A61N 1/05

(52) U.S. Cl. CPC A61N 1/36139; A61N 1/0534

(57) ABSTRACT

Systems and methods for adaptive neuromodulation based on evoked responses are disclosed. An exemplary system comprises at least one lead, an electrostimulator to provide electrostimulation to a neural target, a sensing circuit to sense ERs to electrostimulation, and a controller circuit. In response to electrostimulation delivered to the neural target in accordance with a stimulation setting via a stimulating electrode, the controller circuit can collect sensed ERs to the electrostimulation using at least one sensing electrode on the at least one lead and determine a first stimulation threshold for a stimulation parameter based on a first sensed threshold of the collected sensed ERs. The first stimulation threshold can be a stimulation value at which a change in the collected sensed ERs occurs.

### CLAIM OF PRIORITY

This application claims the benefit of U.S. Provisional Application No. 63/609,147, filed on Dec. 12, 2023, which is hereby incorporated by reference in its entirety.

### TECHNICAL FIELD

This document relates generally to medical systems, and more particularly, but not by way of limitation, to systems, devices, and methods for selective sensing of evoked response and using the same to guide neurostimulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stiimulation (PNS), Functional Electrical Stinulation (FES), and other neural stimulators to treat urinary incontinence, sleep apnea, shoulder subluxation, etc.

A therapy device may be configured to treat a condition. Thus, by way of example and not limitation, a DBS system may be configured to treat motor disorders such as, but not limited to, tremor, bradykinesia, and dyskinesia associated with Parkinson's Disease (PD). In another nonliiting example, a stimulation device, such as neurostimulation device (e.g., DBS, SCS, PNS or TENS), may be configured to treat pain. In another nonlimiting example, a device, such as a myocardial stimulator and/or neurostimulator, may be configured to treat cardiovascular condition. Settings of the therapy device may be programmed based on observed clinical effects so that the therapy provides desirable intended effects (e.g., reduced tremor, bradykinesia, and dyskinesia for a PD therapy, desirable pain relief or paresthesia coverage for a pain therapy, desirable blood pressure and/rhythms for a cardiovascular therapy) while avoiding undesirable side effects.

### SUMMARY

An example (e.g., “Example 1”) of a system may include at least one lead, an electrostimulator, a sensing circuit, and a controller circuit. The at least one lead can include a plurality of electrodes. The electrostimulator can be configured to provide electrostimulation to a neural target of a patient. The neural target can refer to an evoked potential target or a therapeutic stimulation target. An evoked potential target refers to a particular evoked potential that is a response from the patient that is targeted. A therapeutic stimulation target refers to a therapeutic stimulation value at which the stimulation therapy is administered. The sensing circuit can be configured to sense an evoked response (ER) to the electrostimulation. The controller circuit can be operably connected to the electrostimulator and the sensing circuit. The controller circuit can be configured to deliver the electrostimulation to the neural target in accordance with a stimulation setting via at least one stimulating electrode selected from the plurality of electrodes on the at least one lead. The controller circuit can be configured to collect sensed ERs to the electrostimulation using at least one sensing electrode on the at least one lead. The controller circuit can be configured to determine a first stimulation threshold for a stimulation parameter based on a first sensed threshold of the collected sensed ERs. The first stimulation threshold can be a stimulation value at which a change in the collected sensed ERs occurs.

In Example 2, the subject matter of Example 1 may optionally be configured such that the controller circuit is configured to determine a second stimulation threshold for the stimulation parameter based on additional therapy parameters. The second stimulation threshold can indicate a maximum value beyond which side effects occur.

In Example 3, the subject matter of any one of Examples 1-2 may optionally be configured such that the change in the collected sensed ERs comprises a change from increasing ERNA amplitude values to non-increasing ERNA amplitude values.

In Example 4, the subject matter of any one of Examples 1-3 may optionally be configured such that the change in the collected sensed ERs comprises a change from decreasing ERNA frequency values to non-decreasing ERNA frequency values.

In Example 5, the subject matter of any one of Examples 1-4 may optionally be configured such that the controller circuit is further configured to detect an inflection point associated with the first sensed threshold; and the inflection point is detected based on a comparison of a difference in consecutive electrostimulation recordings.

In Example 6, the subject matter of Example 5 may optionally be configured such that the difference in consecutive electrostimulation recordings comprises a comparison of a first-ordered trough of a stimulation wave to a second-ordered positive peak of the stimulation wave.

In Example 7, the subject matter of any one of Examples 1-6 may optionally be configured such that the controller circuit is further configured to deliver electrostimulation therapy to the neural target using a stimulation parameter set above or below the first stimulation threshold to cause a subsequent sensed ER value to be above or below, respectively, the first sensed threshold.

In Example 8, the subject matter of Example 7 may optionally be configured such that the controller circuit is configured to deliver the therapy electrostimulation based on additional sensed parameters and the additional sensed parameters comprise a frequency of at least one of the sensed ERs, an N1-P2 peak amplitude, an N1-P2 peak delay, a size of an evoked potential envelope, or a relative amplitude of each peak.

In Example 9, the subject matter of Example 8 may optionally be configured such that the controller circuit is further configured to use at least one of the additional sensed parameters to determine a state of the patient.

In Example 10, the subject matter of claim 9 may optionally be configured such that the state of the patient comprises one of a sleep state, an awake state, a state of receiving medication, a state of diminishing medication release, sufficient therapy, insufficient therapy, or an experience of at least one side effect.

In Example 11, the subject matter of any one of Examples 1-10 may optionally be configured such that the controller circuit is further configured to update the first stimulation threshold.

In Example 12, the subject matter of any one of Examples 1-11 may optionally be configured such that the controller circuit is further configured to change at least one of a plurality of stimulation parameters associated with delivering electrostimulation to the patient in response to the plurality of stimulation parameters causing the sensed ERs to be below or above the first stimulation threshold.

In Example 13, the subject matter of any one of Examples 1-12 may optionally be configured such that the controller circuit is further configured to perform an initial calibration for the electrostimulation to calibrate the sensed ERs with respect to a state of the patient.

In Example 14, the subject matter of any one of Examples 1-13 may optionally be configured such that the controller circuit is further configured to determine a state of the patient and adjust the plurality of stimulation parameters in association with the determined state.

In Example 15, the subject matter of any one of Examples 1-14 may optionally be configured such that the controller circuit is further configured to recalibrate the first stimulation threshold and adjust the stimulation setting based on the recalibrated first stimulation threshold.

Example 16 includes subject matter (such as a method, means for performing acts, machine readable medium including instructions that when performed by a machine cause the machine to perform acts, or an apparatus to perform). The subject matter may include delivering electrostimulation to a neural target of a patient in accordance with a stimulation setting via a stimulating electrode selected from a plurality of electrodes on at least one lead. The subject matter may include sensing evoked responses (ERs) using sensing electrodes connected to a sensing circuit, the sensing electrodes selected from the plurality of electrodes on the at least one lead. A processing system may be used to determine a first stimulation threshold for a stimulation parameter based on a first sensed threshold of the sensed ERs. The first stimulation threshold can indicate a minimum value or a maximum value for the stimulation parameter. The processing system may be used to determine a second stimulation threshold for the stimulation parameter based on additional therapy parameters. The second stimulation threshold can indicate a maximum value for the stimulation parameter. The processing system can be used to deliver electrostimulation therapy to the neural target using a stimulation parameter set to cause a subsequent sensed ER value to be above the minimum value or below the maximum value and below the second stimulation threshold.

In Example 17, the subject matter of Example 16 may optionally be configured such that the processing system is used to detect a saturation point associated with the stimulation parameter. The saturation point can be detected based on a comparison of a difference in consecutive electrostimulation recordings.

In Example 18, the subject matter of any of Examples 16-17 may optionally be configured such that the difference in consecutive electrostimulation recordings comprises a comparison of a first-ordered trough of a stimulation wave to a second-ordered positive peak of the stimulation wave.

In Example 19, the subject matter of any one of Examples 16-18 may optionally be configured such that the electrostimulation therapy is delivered based on additional sensed parameters; and the additional sensed parameters comprise a frequency of at least one of the sensed ERs, an N1-P2 peak amplitude, an N1-P2 peak delay, a size of an evoked potential envelope, or a relative amplitude of each peak. In some examples, information related to frequency can refer to an instantaneous frequency, an averaged frequency, a Fourier decomposition, wavelet similarities, and/or correlations with frequencies.

In Example 20, the subject matter of Example 19 may optionally be configured such that the processing system is used to use at least one of the additional sensed parameters to determine a state of the patient, wherein the state of the patient comprises one of a sleep state, an awake state, a state of receiving medication, or a state of diminishing medication release, sufficient therapy, insufficient therapy, or an experience of at least one side effect.

In Example 21, the subject matter of any one of Examples 16-20 may optionally be configured such that the processing system is used to update the first stimulation threshold based on a change of state of the patient and/or update the second stimulation threshold based on a change of state of the patient.

In Example 22, the subject matter of any one of Examples 16-21 may optionally be configured such that the processing system is used to recalibrate the first stimulation threshold and the second stimulation threshold; and adjust the stimulation parameter to cause the subsequent sensed ER value to be above a minimum value or a maximum value each associated with the recalibrated first stimulation threshold or below the recalibrated second stimulation threshold

In Example 23, the subject matter of any one of Examples 16-22 may optionally be configured such that the processing system is used to change the stimulation parameters associated with the delivered electrostimulation in response to the stimulation parameters causing the sensed ERs to be below the first threshold or above the second threshold.

In Example 24, the subject matter of any one of Examples 16-23 may optionally be configured such that the processing system is used to determine a state of the patient and adjusting the delivered electrostimulation to a predetermined stimulation value associated with the determined state.

In Example 25, the subject matter of any one of Examples 16-24 may optionally be configured such that the sensing electrodes include two or more electrodes immediately adjacent to the stimulating electrode on the at least one lead.

In Example 26, the subject matter of any one of Examples 16-25 may optionally be configured such that the at least one lead includes a deep brain stimulation (DBS) lead, and wherein the electrostimulator is configured to provide DBS to a brain target of the patient.

In Example 27, the subject matter of any one of Examples 16-26 may optionally be configured such that the processing system is used to determine the first stimulation threshold or the second stimulation threshold based on a particular time period that the electrostimulation is delivered.

In Example 28, the subject matter of any one of Examples 16-27 may optionally be configured such that the processing system is used to determine the first stimulation threshold or the second stimulation threshold based on a particular activity being performed by the patient.

In Example 29, the subject matter of any one of Examples 16-28 may optionally be configured such that the processing system is used to provide stimulation values for the stimulation parameter set based on at least one of a time of day that the electrostimulation and a particular activity being performed by the patient.

Example 30 includes subject matter (such as a method, means for performing acts, machine readable medium including instructions that when performed by a machine cause the machine to perform acts, or an apparatus to perform). The subject matter may include delivering electrostimulation to a neural target of a patient in accordance with a stimulation setting via a stimulating electrode selected from a plurality of electrodes on at least one lead. The electrostimulation can be delivered to the neural target of the patient via a medical-device system that comprises an electrostimulator and the at least one lead coupled thereto. The subject matter may include sensing evoked responses (ERs) using sensing electrodes connected to a sensing circuit, the sensing electrodes selected from the plurality of electrodes on the at least one lead. The subject matter may include determining a first stimulation threshold for a stimulation parameter based on a first sensed threshold of the sensed ERs. The first stimulation threshold can be a stimulation value at which a change in the sensed ERs occur. The subject matter may include determining a second stimulation threshold for the stimulation parameter based on additional therapy parameters. The second stimulation threshold can indicate a maximum value for the stimulation parameter. The subject matter may include delivering electrostimulation therapy to the neural target using a stimulation parameter set to cause a subsequent sensed ER value to be above or below the first stimulation threshold and below the second stimulation threshold.

In Example 31, the subject matter of Example 30 may optionally be configured such that the electrostimulation therapy is delivered based on additional sensed parameters. The additional sensed parameters can comprise a frequency of at least one of the sensed ERs, an N1-P2 peak amplitude, an N1-P2 peak delay, a size of an evoked potential envelope, or a relative amplitude of each peak.

In Example 32, the subject matter of any one of Examples 31 may optionally be configured such that at least one of the additional sensed parameters can be used to determine a state of the patient, wherein the state of the patient comprises one of a sleep state, an awake state, a state of receiving medication, or a state of diminishing medication release, sufficient therapy, insufficient therapy, or an experience of at least one side affect.

In Example 33, the subject matter of any one of Examples 30-32 may optionally be configured such that the first stimulation threshold is updated based on a change of state of the patient.

In Example 34, the subject matter of any one of Examples 30-33 may optionally be configured such that the second stimulation threshold is updated based on a change of state of the patient.

In Example 35, the subject matter of any one of Examples 30-34 may optionally be configured such that at least one of a plurality of stimulation parameters associated with the delivered electrostimulation is changed in response to the stimulation parameters causing the sensed ERs to be below or above the first sensed threshold or above the second sensed threshold.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates, by way of example and not limitation, an example of a neuromodulation system configured to adjust neuromodulation therapy and device programming based on selected evoked responses.

FIG. 8A illustrates, by way of example and not limitation, distributions of neuromodulation parameters associated with amplitude.

FIG. 8B illustrates, by way of example and not limitation, distributions of neuromodulation parameters associated with frequency.

FIG. 9 illustrates, by way of example and not limitation, a method of adaptive neuromodulation based on evoked responses (ERs).

FIG. 10 illustrates, by way of example and not limitation, a neuromodulation therapy system, which may be used to deliver DBS.

FIG. 11 illustrates, by way of example and not limitation, an example of a method for neuromodulation therapy calibration and operation.

FIGS. 12A-12B illustrate, by way of example and not limitation, an example of distributions associated with evoked response features.

FIG. 13 illustrates, by way of example and not limitation, an example of a method for saturation detection.

FIGS. 14A-14B illustrate, by way of example and not limitation, saturation distributions for detecting saturation.

FIGS. 15A-15C illustrate, by way of example and not limitation, saturation distributions including maxima for detecting saturation.

FIG. 16 illustrates, by way of example and not limitation, distributions of ER dynamics at a particular DBS intensity.

FIG. 17 illustrates, by way of example and not limitation, an example of a method for saturation detection.

FIG. 18 illustrates, by way of example and not limitation, an example of a method for saturation detection.

FIG. 19 illustrates, by way of example and not limitation, an example of a method for recalibration a saturation point.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There can be differing effectiveness of neuromodulation therapy depending on a patient's state, e.g., vary depending on a medication level, a physical state (sleep, awake, physical activity, etc.), condition or disorder, anatomy (anatomical target), or symptoms to improve (e.g., tremor to cognitive skill improvement). States can also include an active state vs. inactive state. States can also include a “Therapy ON” or “Effective Therapy” state, which in the context of DBS can be a combination of medication and stimulation. The following description can include determining effective stimulation parameters without and/or with medication being administered. Further, the effective stimulation parameters can be determined in relationship with the medication wearing off or waxing/waning over a day of dosing. Adjusting the neuromodulation therapy to these conditions can provide for more effective treatment.

Evoked responses (ERs) may be used to determine these states or conditions of the patient. The programming of device settings (e.g., sensing parameters or neurostimulation parameters) can be adjusted and stored in relation to these states or conditions and used in subsequent administration of treatment in order to find more effective treatment more quickly. The ERs may be caused by stimulation that provides ERs, stimulation that provides therapy, and stimulation that can both deliver therapy and provide ERs. More specifically, features of the ERs can also be used to determine the state or condition of the patient. Examples of the signal features can include a signal amplitude, magnitude, peak value, value range, a signal curve length, or a signal power or RMS value of an ER signal within a time window, such as the epoch-averaged ERs, N1-P2 peak amplitude, N1-P2 delay (distance on the x-axis in recording time, which can be a measure of the period of decaying sine wave), size of evoked potential envelope, relative amplitudes of each peak (distance between any given peak and the midpoint).

The features of the ERs can be used to determine a minimum and/or a maximum therapy threshold for neuromodulation treatment in order to avoid levels of stimulation that create discomfort or side effects and in order to avoid levels that are below a threshold of effective treatment (e.g., to maximize therapy and/or minimize/counter side effects). This minimum or maximum therapy threshold can be used to maintain therapy above an ineffective therapy level and within an effective range of therapy and can vary based on whether medication is being administered and/or whether stimulation is being administered or increased/decreased. Additionally therapy parameters can be used to ensure effective stimulation and in order to continue to provide effective stimulation, even in the midst of changes to the state of the patient. In addition, the minimum and/or maximum therapy thresholds can be used to maintain the therapy within an effective range of therapy without increasing beyond a threshold that may cause side effects. These variations can be monitored and/or maintained throughout decreasing or waxing/waning medication effectiveness throughout a day with medication dosing. Responses may be modulated by the details of the sensing, including amplifier settings, relationships between stimulating and sensing electrodes, natures of stimulating or sensing electrodes including geometry and surface among other factors, and signal processing occurring during and after measurement, including treatment within analogue or digital hardware, firmware, or software.

This disclosure refers to an ERNA caused by electrostimulation, as a nonlimiting example of an ER to electrostimulation provided by an electrostimulator. The present subject matter may be applied for other ERs to other electrostimulation. The electrostimulation may be therapeutic in nature in some examples, or diagnostic in nature in others.

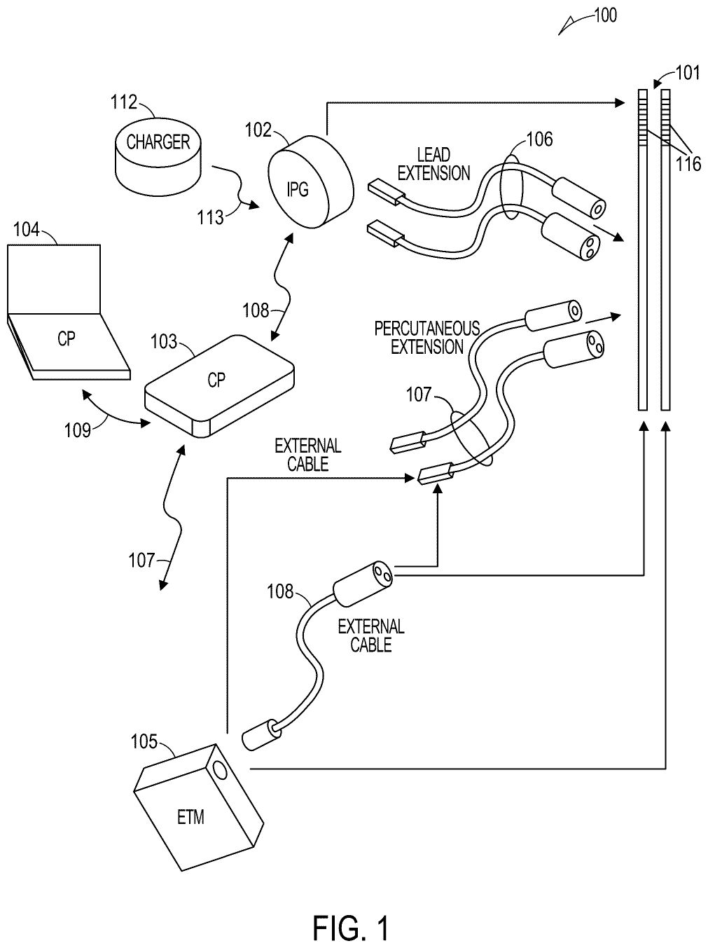

FIG. 1 illustrates, by way of example and not limitation, an electrical stimulation system 100, which may be used to deliver DBS. The electrical stimulation system 100 may generally include one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

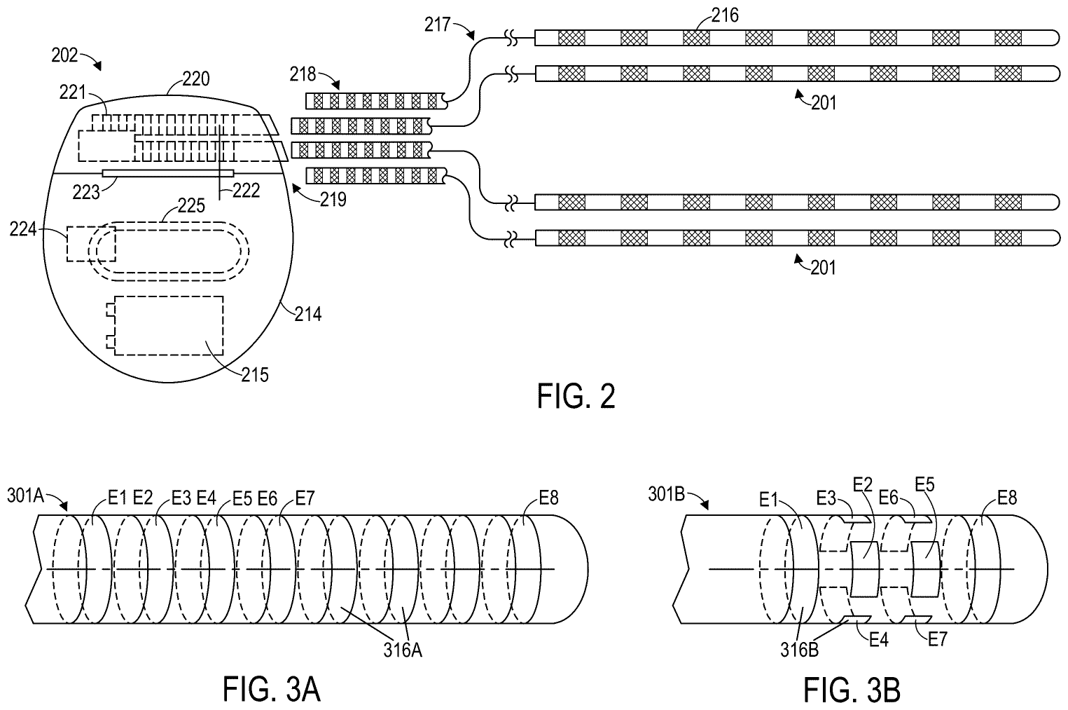

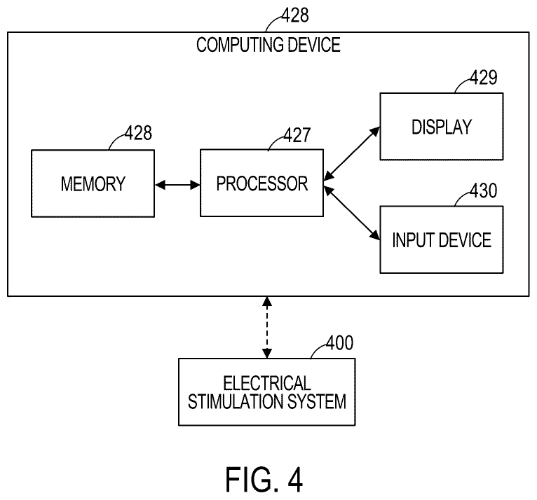

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more rows of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also includes a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide more-superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

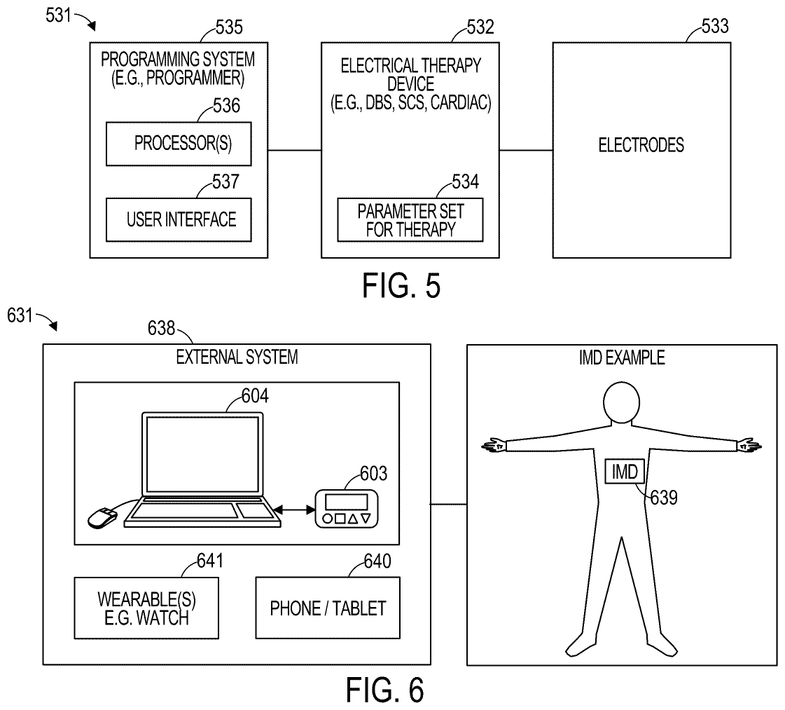

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

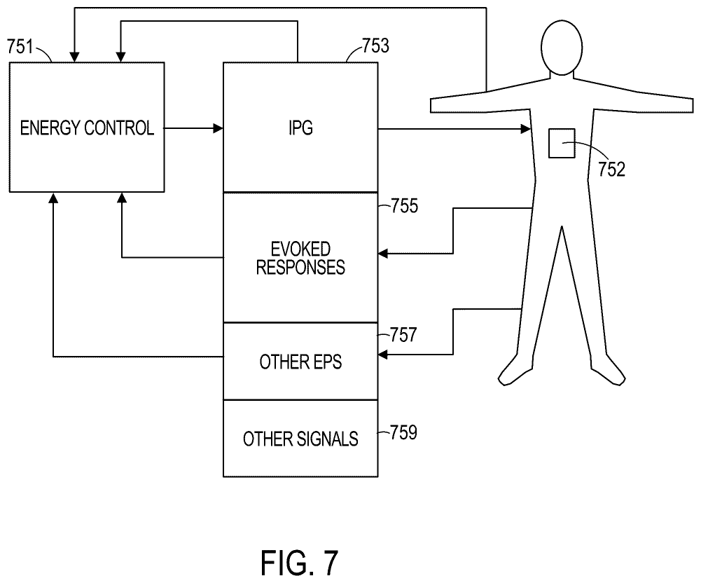

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameter sets through a computerized programming system to allow the optimum modulation parameters to be determined based on patient feedback or other means and to subsequently program the desired modulation parameter sets.

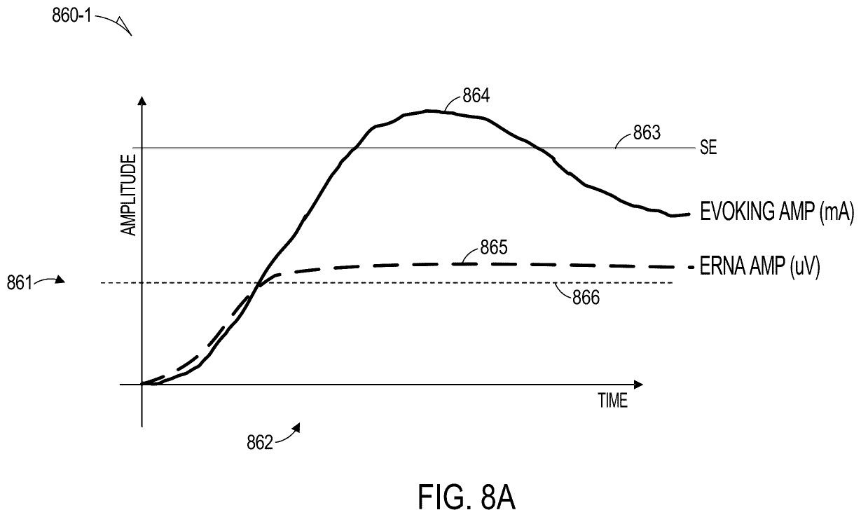

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

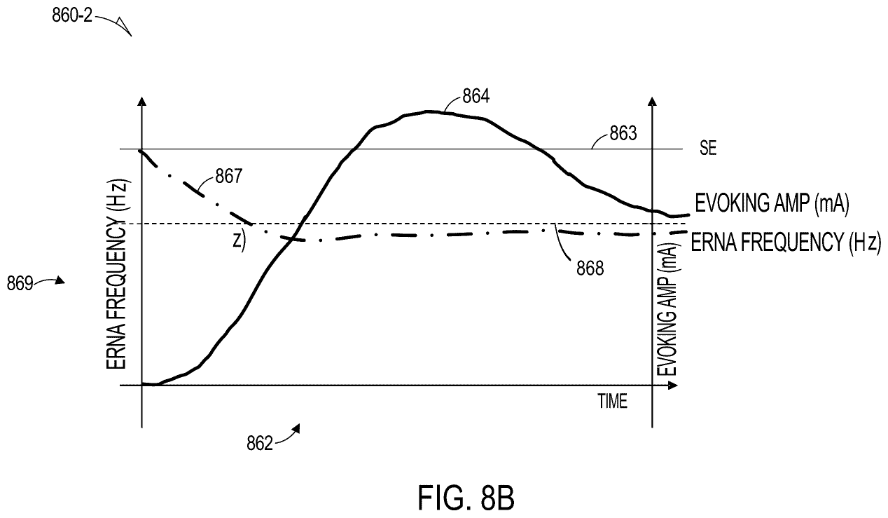

FIG. 7 illustrates, by way of example and not limitation, an example of a neuromodulation system configured to adjust neuromodulation therapy and device programming based on selected evoked responses. The illustrated system in FIG. 7 includes components including an energy control unit 751, an implantable pulse generator (IPG) 753, evoked responses 755, other evoked potentials (EPs) 757, other signals 759, and an implantable medical device (IMD) 752 implanted within a patient. The IMD 752 can provide various ERs 755, other Eps 757, and other signals 759 caused by various stimulation, sensors, etc., to provide for input to the energy control unit 751. The energy control unit 751 can be a device that includes hardware, software, and/or firmware used to process the received information and to provide instructions to the IPG 753 to provide neurostimulation to the patient through the IMD 752. The ERs 755, other Eps 757, and other signals 759 can be used to determine different stimulation and therapy thresholds, both minimums and maximums, as will be described herein, to provide effective therapy to the patient in a variety of different states and conditions.

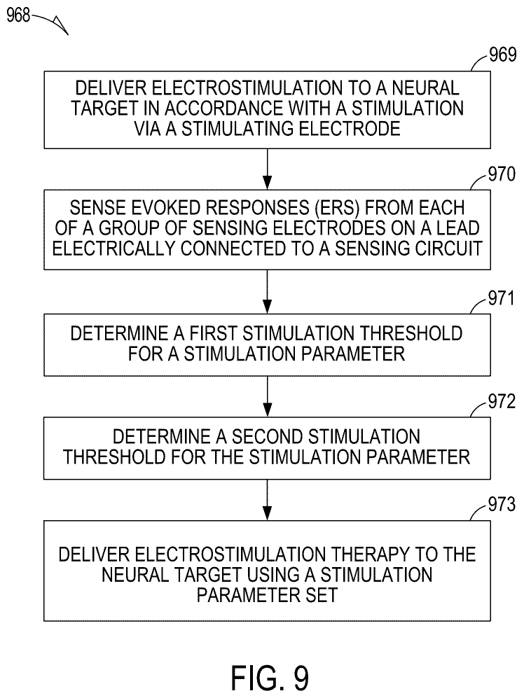

FIG. 8A illustrates, by way of example and not limitation, distributions of neuromodulation parameters associated with amplitude. The distributions 860-1 are shown along an x-axis representing time 862 and a y-axis representing amplitude 861. The distributions 860-1 can include an ERNA amplitude (“ERNA AMP”) 865 (in uV) and an evoking amplitude (“EVOKING AMP”) 864 (in mA). The ERNA amplitude 865 can be an ERNA feature that is used to locate a minimum effective amplitude 866. In this way, the ERNA amplitude 865 can be used as a minimum amplitude for providing neuromodulation therapy. The ERNA amplitude 865 can indicate a particular amplitude at which the ERNA signal plateaus and no longer increases. For example, as the evoking amplitude 864 increases, the ERNA amplitude 865 increases to a difference in the rate of change of the ERNA amplitude 865 resulting in a plateau or flattening of the ERNA amplitude 865. At this difference in the rate of change, the minimum amplitude corresponding to the minimum effective amplitude 866 can be used as a baseline or stimulation threshold. The ERNA amplitude 865 can indicate a particular amplitude at which the ERNA signal plateaus and no longer increases. The ERNA amplitude 865 can be a boundary between ineffective and effective neural stimulation. This minimum threshold can be variable, however, depending upon a state or condition of the patient. For example, a medication state of the patient, such as a low medication state (when medication is washing out or decreasing below a particular threshold in the patient's body) or a high medication state (when medication is being administered to or absorbed by the patient and is increasing), can affect the amplitude value of this effective minimum amplitude 866. Therefore, the ERNA minimum 865 can be tested and indicate this change.

Further, the evoking amplitude 864 can be above a side effect threshold 863 that indicates the patient is experiencing side effects at a particular level that may bring discomfort. The side effect threshold 863 can indicate a boundary between no and some (e.g., meaningful) side effects experienced by the patient. In some examples, the discomfort and additional side effects do not result in increased therapy effectiveness. For this reason, the evoking amplitude 864 can be kept below this side effect threshold 863 but also above the ERNA minimum in order to provide effective therapy. Additional parameters and/or signals can be used in combination with the ERNA amplitude 865 and/or side effect threshold 863 to determine a best amplitude for a particular state or condition of the patient. These minimum and maximum amplitude values can be recalibrated and tested at particular time intervals, during particular changes of state or condition, etc., in order to maintain a proper range for the amplitude based on any changes in or to the patient.

In some examples, the effective minimum amplitude can be a first stimulation threshold. The first stimulation threshold can be a stimulation value at which a change in the collected sensed ERs occurs. For example, the change in the collected sensed ERs comprises a change from increasing ERNA amplitude values (e.g., increasing ERNA amplitude 865 illustrated in FIG. 8A) to non-increasing ERNA amplitude values (e.g., when the ERNA amplitude 865 plateaus and levels out). This change can indicate an amplitude level that provides effective therapy and a level at which to maintain the amplitude value above. In some examples, a second stimulation threshold can be determined for the stimulation parameter based on additional therapy parameters. The second stimulation threshold can indicate a maximum value, such as side effect threshold 863 beyond which side effects occur.

In some examples, the ERNA amplitude 865 can include a first significant increase of the ERNA amplitude values and a second insignificant increase of the ERNA amplitude values. For example, a first amount of increase may be significant and indicate a particular inflection point of change of rate of increase. Further, a second amount of increase may not be significant and may not indicate an inflection point and be below a particular threshold rate of increase. The inflection point or significant increase can be used to indicate the amplitude level that provides effective therapy. The second rate of change can be less than the first rate of change. In one example, the difference in a rate of change can be accepted by a threshold of +5 uV/mA for the first rate of change and a difference of +3 uV/mA for the second rate of change can be insufficient. However, a rate of change of +10 uV/mA followed by a rate of change of +1 uV/mA can be accepted as indicating a state change.

The first stimulation threshold can be a threshold at which the ERNA responses indicate whether the therapy is OFF or ON. Additional information (such as other ERs, other signals, etc.) can be used, along with additional logic, to check for the therapy floor or minimum threshold at particular intervals of time (e.g., every 5 minutes, 10 minutes, etc.) and continue to increase as needed to maintain the therapy above the floor.

FIG. 8B illustrates, by way of example and not limitation, distributions of neuromodulation parameters associated with frequency. The distributions 860-2 are shown along an x-axis representing time 862 and a y-axis representing frequency 869. The distributions 860-2 can include an ERNA frequency 867 (in Hz) and an evoking amplitude (“EVOKING AMP”) 864 (in mA). The ERNA frequency 867 can be an ERNA feature that is used to locate a minimum effective amplitude 868. In this way, the ERNA frequency 867 can be used to determine a minimum amplitude for providing neuromodulation therapy. The ERNA frequency 867 can indicate a particular amplitude at which the ERNA signal plateaus and no longer increases. For example, as the evoking amplitude 864 increases, the ERNA frequency 867 decreases to a difference in the rate of change of the ERNA frequency 867 resulting in a plateau or flattening of the ERNA frequency 867. At this difference in the rate of change, the minimum amplitude corresponding to the minimum effective amplitude 868 can be used as a baseline or stimulation threshold. As an example, the ERNA frequency 867 can be a boundary between ineffective and effective neural stimulation. This minimum threshold can be variable, however, depending upon a state or condition of the patient. For example, a medication state of the patient, such as a low medication state (when medication is washing out or decreasing below a particular threshold in the patient's body) or a high medication state (when medication is being administered to or absorbed by the patient and is increasing), can affect the amplitude value of this effective minimum amplitude 866. Therefore, the ERNA minimum 865 can be tested and indicate this change.

Further, the evoking amplitude 864 can be above a side effect threshold 863 that indicates the patient is experiencing side effects at a particular level that may bring discomfort. The side effect threshold 863 can indicate a boundary between no and some (e.g., meaningful) side effects experienced by the patient. In some examples, the discomfort and additional side effects do not result in increased therapy effectiveness. For this reason, the evoking amplitude 864 can be kept below this side effect threshold 863 but also above the ERNA minimum in order to provide effective therapy. Additional parameters and/or signals can be used in combination with the ERNA frequency 867 and/or side effect threshold 863 to determine a best stimulation amplitude for a particular state or condition of the patient. These minimum and maximum amplitude values can be recalibrated and tested at particular time intervals, during particular changes of state or condition, etc., in order to maintain a proper range for the amplitude based on any changes in or to the patient.

In some examples, similar to the amplitude change, the ERNA frequency 867 can include a first significant decrease of the ERNA frequency values and a second insignificant decrease of the ERNA frequency values. For example, a first amount of decrease may be significant and indicate a particular inflection point of change of rate of increase. Further, a second amount of decrease may not be significant and may not indicate an inflection point and be below a particular threshold rate of increase. The inflection point or significant increase can be used to indicate the frequency level that provides effective therapy. The second rate of change can be less than the first rate of change.

In some examples, the effective minimum amplitude 868 can be a first stimulation threshold. The first stimulation threshold can be a stimulation value at which a change in the collected sensed ERs occurs. For example, the change in the collected sensed ERs comprises a change from decreasing ERNA frequency values (e.g., decreasing ERNA frequency 867 illustrated in FIG. 8B) to non-decreasing ERNA frequency values (e.g., when the ERNA frequency 867 plateaus and levels out). In some examples, a second stimulation threshold can be determined for the stimulation parameter based on additional therapy parameters. The second stimulation threshold can indicate a maximum value, such as side effect threshold 863 beyond which side effects occur.

A recalibration of the first stimulation threshold and/or the second stimulation threshold can be performed in order to adjust for changing patient states and/or changing stimulation parameters or factors. The recalibration can be performed on a schedule where the minimum effective amplitude threshold is determined and then, using a heuristic, a therapeutic amplitude (above the minimum, e.g., +20%) can be determined to set the stimulation to. In this way, the stimulation parameter can be adjusted to provide effective therapy but not provide too much stimulation therapy.

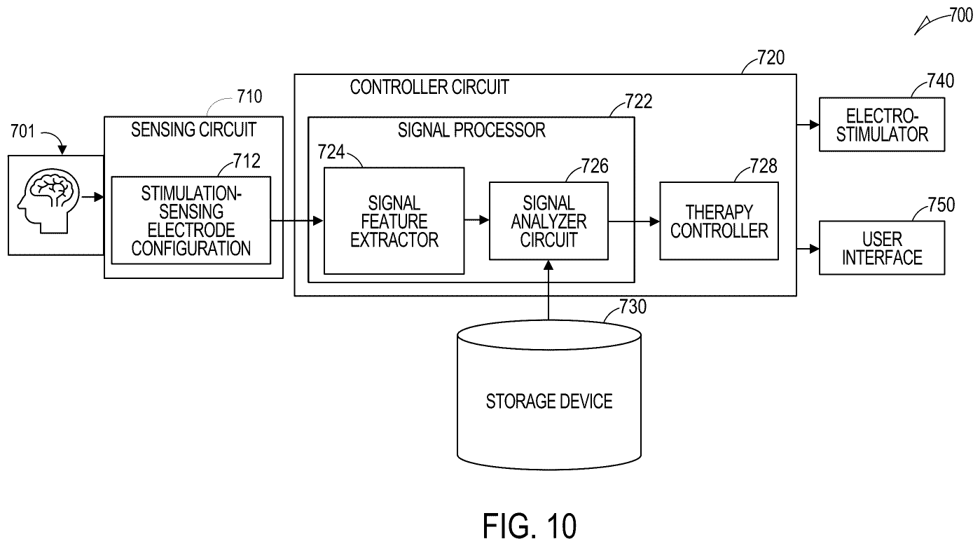

FIG. 9 illustrates, by way of example and not limitation, a method of adaptive neuromodulation based on evoked responses (ERs). The method 968 can include, at 969, delivering electrostimulation to a neural target in accordance with a stimulation setting via a stimulating electrode. The stimulating electrode can be selected from a plurality of electrodes on at least one lead. The electrostimulation therapy can be delivered based on additional sensed parameters. The additional sensed parameters can include a frequency of at least one of the sensed ERs, an N1-P2 peak amplitude, an N1-P2 peak delay, a size of an evoked potential envelope, or a relative amplitude of each peak. At least one of the additional sensed parameters can be used to determine a state of the patient. The state of the patient can include one of a sleep state, an awake state, a state of receiving medication, or a state of diminishing medication release, sufficient therapy, insufficient therapy, or an experience of at least one side effect.

The method 968 can include, at 970, sensing evoked responses (ERs) using sensing electrodes connected to a sensing circuit, the sensing electrodes selected from the plurality of electrodes on the at least one lead. The sensing electrodes can include two or more electrodes immediately adjacent to the stimulating electrode on the at least one lead.

The method 968 can include, at 971, using a processing system to determine a first stimulation threshold for a stimulation parameter based on a first sense threshold of the sensed ERs. The first stimulation threshold can indicate a minimum value or a maximum value for the for the first sensed threshold. The maximum value can be associated with the ERNA amplitude value (e.g., ERNA amplitude 865) illustrated in FIG. 8A. The minimum value can be associated with the ERNA frequency value (e.g., ERNA frequency 867) illustrated in FIG. 8B. The first stimulation threshold can be updated based on a change of state of the patient.

The method 968 can include, at 972, using the processing system to determine a second stimulation threshold for the stimulation parameter based on additional therapy parameters. The second stimulation threshold can indicate a maximum value for the sensed ERs. The second stimulation threshold can be updated based on a change of state of the patient. The first stimulation threshold and/or the second stimulation threshold can be determined based on a particular time period that the electrostimulation is delivered. The first stimulation threshold and the second stimulation threshold can be determined based on a particular activity being performed by the patient.

The method 968 can include, at 973, using the processing system to deliver electrostimulation therapy to the neural target using a stimulation parameter set to cause a subsequent sensed ER value to be above the first value and below the second value. At least one of the stimulation parameters associated with the delivered electrostimulation can be changed in response to the stimulation parameters causing the sensed ERs to be below the first stimulation threshold or above the second stimulation threshold. In some examples, the stimulation parameters can be adjusted in order to cause the sensed ERs to move further in the direction of or more towards the first or second stimulation threshold, respectively. A state of the patient can be determined and the delivered electrostimulation can be adjusted to a predetermined stimulation value associated with the determined state.

The method 968 can include using the processing system to detect a saturation point associated with the stimulation parameter. The saturation point can be detected based on a comparison of a difference in consecutive electrostimulation recordings. The detection of the difference in consecutive electrostimulation recordings can include a comparison of a first-ordered trough of a stimulation wave to a second-ordered positive peak of the stimulation wave.

The method 968 can include providing stimulation values for the first stimulation parameter and the second stimulation parameter based on previous determinations of the first stimulation parameter and the second stimulation parameter. The previous determinations can be based on at least one of a time of day that the electrostimulation is performed and a particular activity being performed by the patient.

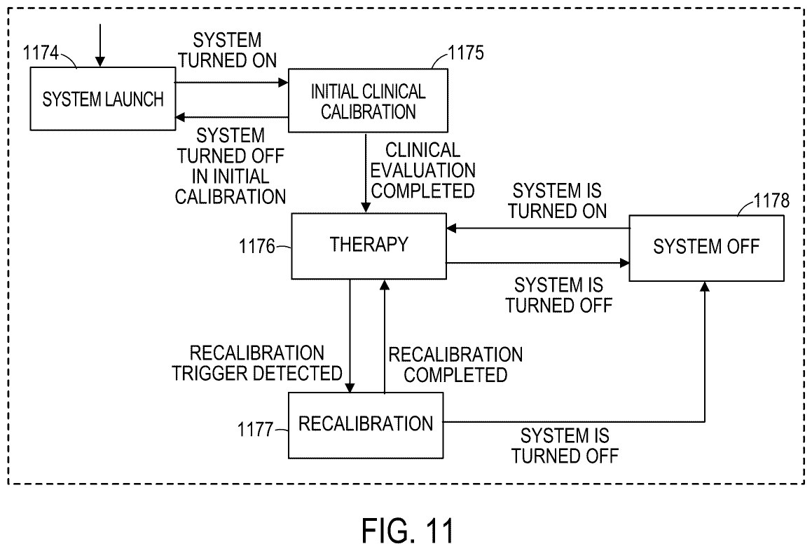

FIG. 10 illustrates, by way of example and not limitation, a neuromodulation therapy system, which may be used to deliver DBS based on evoked responses. The system 1000 includes a sensing circuit 1010, a controller circuit 1020, a storage device 1030, an electrostimulator 1040, and a user interface 1050. Portions of the system 1000 may be implemented in the IPG 102 or the CP 104.

The sensing circuit 1010 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 1040. The ring electrodes and/or the segmented electrodes may be configured as sensing electrodes for sensing ERs, or as stimulating electrodes for delivering electrostimulation pulses. The sensing circuit 1010 may sense ERs from one or more sensing electrodes on a lead placed at target issue (e.g., STN) of a patient 1001 in response to electrostimulation pulses delivered from a stimulating electrode at a stimulation site (e.g., a brain target). The ERs may be sensed in accordance with a stimulating-sensing electrode configuration 1012.

The controller circuit 1020 can include circuit sets comprising one or more other circuits or sub-circuits, such as a signal processor 1022 and a therapy controller 1028. The signal processor 1022 may further include a signal feature extractor 1024 and a signal analyzer circuit 1026. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 1020 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

The signal feature extractor 1024 may extract a signal feature from the filtered ER signal (e.g., the ER signal with the stimulation artifact removed). Examples of the signal features can include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length can be measured as accumulated signal value differences of the sensed evoked response (or an epoch-averaged evoked response) over consecutive unit times (e.g., consecutive data sampling intervals) within the time window. The signal power can be measured as an area under the curve (AUC) of the sensed evoked response (or the epoch-averaged evoked response) within the time window. In some examples, the signal analyzer circuit 1026 may generate a spatial distribution of extracted signal features across sensing locations of the sensing electrodes, such as the selected group of sensing electrodes as illustrated in FIG. 10.

The signal analyzer circuit 1026 may compare the filtered ERs, or signal features or a spatial distribution of signal features derived therefrom, to one or more states or conditions of the patient. In some examples, the states or conditions are used to adjust the neuromodulation parameters. In some examples, the signal analyzer circuit 1026 may accumulate the sensed ERs, and therefore determined states or conditions, obtained in multiple stimulation-ER recording sessions during which stimulation pulses are delivered via a particular stimulating electrode with varying stimulation parameter settings (e.g., stimulation amplitude, frequency, or pulse width), and compare the accumulated ERs (or signal features or a spatial distribution of signal features derived from the filtered ERs) to previously stored states or conditions. In an example, the states or conditions of the patient can be user-provided or loaded into the system for easy recognition. In another example, the state or condition can be associated with a target ER or target ER feature template representing a patient-specific ER feature or a population-based ER feature to electrostimulation of the neural target. In an example, the target ER template may be used to adjust the neuromodulation therapy to relieve symptoms or other goals such as co-therapy (e.g., leads that inject drugs or light), and side-effect avoidance. The signal analyzer circuit 1026 may determine a distribution of sensed ER features, compare the determined distribution of sensed ERs to the corresponding states or conditions to determine whether to adjust the neuromodulation therapy.

The therapy controller 1028 can generate a control signal to the electrostimulator 1040 to adjust the thresholds of the neuromodulation therapy based on the determined state or condition. The electrostimulator 1040 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 1028 can be implemented as a proportional integral (PI) controller, a proportional-integral-derivative (PID) controller, or other suitable controller that takes the comparison of the sensed ERs (or features or a distribution of the features thereof) to the corresponding states or conditions as a feedback on the adjustment of stimulation settings.

The electrostimulator 1040 can be an implantable module, such as incorporated within the IPG 10. Alternatively, the electrostimulator 1040 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 1040 can deliver stimulation in accordance with the adjusted therapy setting.

In some examples, the therapy controller 1028 can generate a recommendation to the user to adjust the device setting (e.g., a programmable parameter of the electrostirmuilator 1040) to cause the sensed ERs to align with or to compare more favorably to one or more states or conditions of the patient. In some embodiments, the display may provide a suggestion to the user to adjust stimulation parameters to cause the since developed responses to more favorably compare to the state or condition or a transition to a different state or condition. In some examples, the therapy controller 1028 may determine or modify therapeutic stimulation settings based on the sense ERs or features thereof and the corresponding determined state or condition of the patient. The electrostimulator 1040 may deliver therapeutic stimulation (e.g., DBS) in accordance with the determined or modified therapeutic stimulation settings.

In some examples, the user interface 1050 allows a physician to remotely review therapy settings and treatment history, consult with the patient to obtain information including pain relief and SCS-related side effects or symptoms, perform remote programming of the electrostimulator 1040, or provide other treatment options to the patient. The user interface 1050 can allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program can include a set of stimulation parameters with respective pre-determined values. In some examples, the user interface 1050 can include a display to display textually or graphically information provided by the user via an input unit, and device settings including, for example, feature selection, sensing configurations, signal pre-processing settings, therapy settings, optionally with any intermediate calculations. In an example, the user interface 1050 may present to the user an “optimal” or improved therapy setting, such as determined based on a closed-loop or adaptive feedback control of electrostimulation based on a selected evoked response signal feature, in accordance with various embodiments discussed in this document. In some examples, the user can use the user interface 1050 to provide feedback on a neuromodulation therapy, including, for example, side effects or symptoms arise or persist associated with the neurostimulation, or severity of the symptom or a side effect.

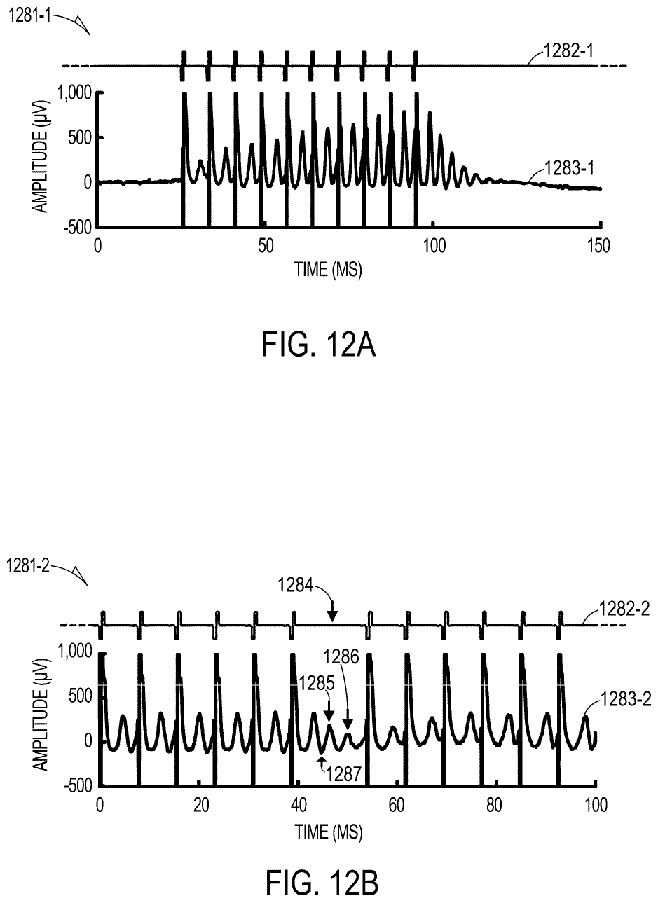

FIG. 11 illustrates, by way of example and not limitation, an example of a method for neuromodulation therapy calibration and operation. The method 1180 can be initialized, at 1174, at a system launch. The system launch can occur upon implantation of an IMD into the patient. The system launch is separate from the system off 1178 (described below) and can be defined as the initial state with no patient information collected or saved. At the launch of the program, and if the system is turned off during the Initial Calibration 1175, the system defaults to this state. The system launch can occur at a particular point in time when a patient is in a state for effective initialization of the IMD system.

In response to the system being launched, the system can be turned on and an initial clinical calibration, at 1175, can occur. The initial clinical calibration 1175 includes ramping current from zero. The initial clinical calibration 1175 identifies clinically significant features within the recorded evoked potentials or ER signals (such as the saturating point, described below). The initial clinical calibration 1175 performs an initial data-driven prediction of optimal therapy settings (for example, intensity, optimal delivery/sensing contact, stimulation frequency, etc.). The initial clinical calibration 1175 can include performing a series of neuromodulation operations, such as neurostimulation to evoke responses, in order to determine a patient's particular response and begin to determine an ER and its associated state or condition for that patient. The system can be turned off in the initial calibration and be shut down or paused until the system launch is performed again. Otherwise, the clinical evaluation for the initial clinical calibration can be completed and therapy, at 1176, can begin.

The therapy 1176 can begin based on a determined minimum amplitude threshold or minimum ERNA feature and/or a maximum threshold amplitude associated with side affects. The neuromodulation therapy 1176 can be maintained within this range between the minimum and maximum thresholds based on the ER values received. The therapy 1176 can include delivering a set of predicted optimal settings corresponding to internal settings adjusted based on previously recorded ERs.

At this stage, the system can be turned off, at 1178, from administering therapy and turned back on to administer therapy 1176. The system may be turned off in response to an electronic error that interferes with proper therapy. The system may be turned off until official therapy is to begin as the initial calibration may occur in a clinical setting as a pre-therapy process prior to a start of the therapy in order to collect data and prepare the system for proper therapy in the future. While no therapy is delivered while the system is off, information regarding the patient and predictions of optimal therapy are saved.

During therapy, at 1176, a recalibration trigger can be detected causing a recalibration 1177. The recalibration trigger can be in response to a change in a state or condition of the patient. The recalibration trigger can be based on a time interval or set time periods for testing of the therapy to ensure the therapy is effective. The recalibration trigger can occur in response to an error or a malfunction where recalibration of the system is necessary to ensure proper therapy to the patient. The recalibration 1177 can update the internal settings related to the patient state or condition based on features recorded from the ERs. These updated internal settings are used to compute a predicted optimal therapy setting that is then delivered in the therapy state, at 1176. In some examples, the recalibration 1177 can cause the system to be turned off, at 1178. In these examples, the recalibration may determine that proper therapy is not possible at this time. The recalibration 1177 may also determine that a state or condition of the patient makes it difficult to provide proper therapy. For example, the state of the patient may indicate that the patient has high levels of medication and no electrostimulation therapy is needed at this time so the system can turn off and restart at a given interval to determine whether the medication state of the patient has changed.

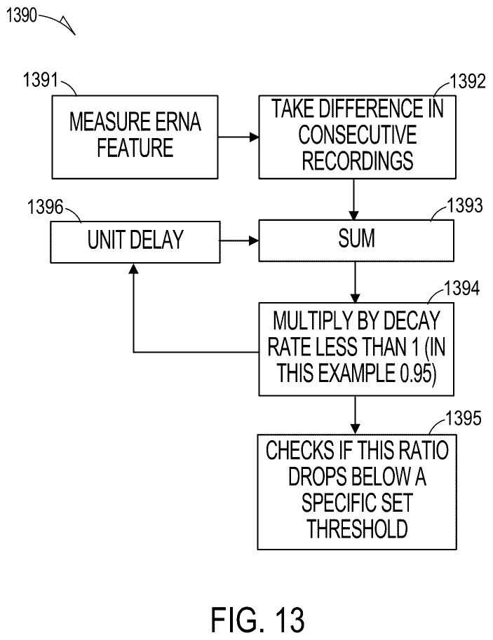

FIGS. 12A-12B illustrate, by way of example and not limitation, an example of distributions associated with evoked response features. The distributions 1281-1, 1281-2 each illustrate an affect that a corresponding generated stimulation 1282-1, 1282-2 has on the patient. For example, FIG. 12A illustrates that the stimulation 1282-1 causes ER signals 1283-1 where each subsequent secondary amplitude peak between primary amplitude peaks of the ER signals 1283-1 are steadily increasing in response to each pulse of the stimulation, showing a building or summation effect. The primary amplitude peak refers to a peak caused in close proximity to the stimulation and a secondary amplitude peak refers to a peak due to a lagging effect of the ER signal behavior. These secondary amplitude peak increases can build in response to the pulses being a particular distance from a previous and subsequent pulse, thereby causing a higher and higher amplitude peak per pulse, as shown. Likewise, at the cessation of the pulses, the amplitude peaks and overall amplitude of the ER signals 1283-1 quickly decline. These secondary amplitude peaks can be at least one ER feature used to determine a state or condition of the patient.

FIG. 12B illustrates an example where a gap or time delay 1284 between stimulation pulses of the stimulation 1282-2 causes a lagging decay effect in the ER signals 1283-2. For example, the secondary amplitude peaks 1285 and 1286 show a decline in amplitude peak over time as the time delay 1284 between stimulation pulses is increased. This decay effect can be an ER feature used to determine a state or condition of the patient for purposes of neuromodulation therapy.

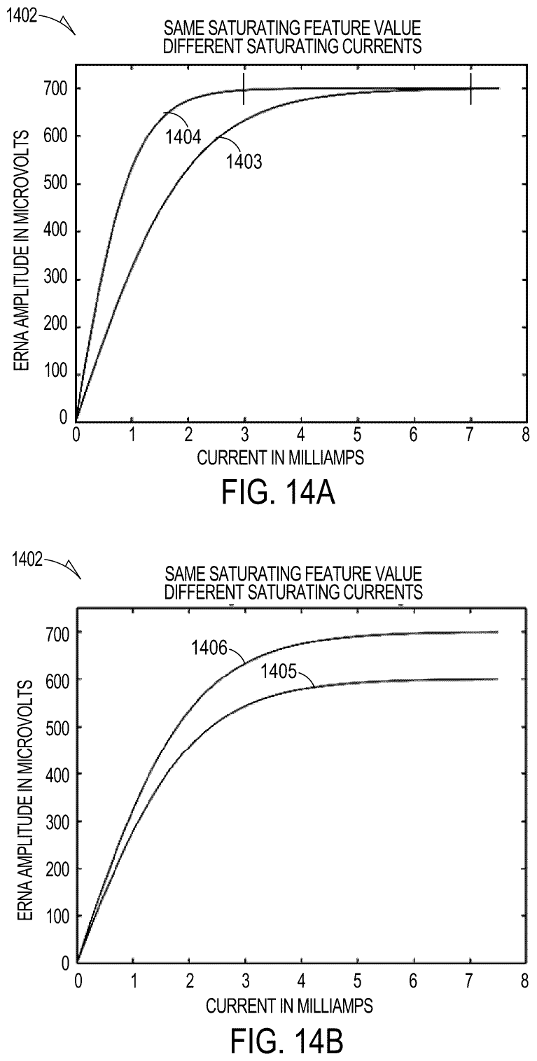

FIG. 13 illustrates, by way of example and not limitation, an example of a method for saturation detection. At 1391, the method 1390 can include measuring an ERNA feature. ER feature(s) may be extracted from the raw ERNA measurement data. For example, features such as amplitude, magnitude, first peak, width, RMS value, and the like may be extracted from the raw ER signals. These features can then be measured.

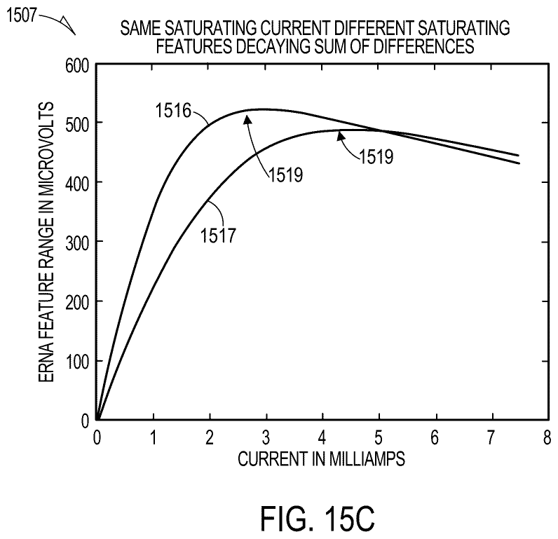

At 1392, a difference in consecutive recordings can be determined. As an example of one difference in consecutive recordings, the difference in the peaks of secondary amplitude peaks 1285 and 1286 in FIG. 12A can be determined. In some examples, the consecutive recordings can refer to not only the consecutive peaks but also to consecutive stimulation windows. Further, a comparison of a first amplitude (such as amplitude 1285 in FIG. 12B) and a second amplitude (such as amplitude 1283-2 in FIG. 12B) could be performed. Many such differences in a plurality of recordings can be summed, at 1393. The summation of these differences in recordings can be multiplied by a decay rate less than 1 (which in this example can be 0.95, however, examples are not so limited). An example of these decaying sum of differences is found in FIG. 15A, further described below, where a decaying sum of differences 1514 is illustrated in diagram 1507. At 1395, a check can be performed to determine if the value of the sum multiplied by the decay rate is less than a specific set threshold. A unit delay can be introduced, at 1396, to allow a set period of time to pass and additional differences in consecutive recordings to be summed in order to allow the value to get closer to the saturation point if the value is not already there. In response to the value being below the specific set threshold, a saturation point can be determined and recorded.

The saturation point can be correlated with the actual corresponding ERNA feature value (such as ERNA features 1513 shown in FIG. 15A) in order to extrapolate the saturation value for the ERNA feature from the calculation based on the sum of differences of consecutive recordings. The saturation point indicates a value beyond which increasing the stimulation parameters has no meaningful effect on therapy. Increasing the stimulation parameters beyond the saturation point may lead to side effects and discomfort in the patient.

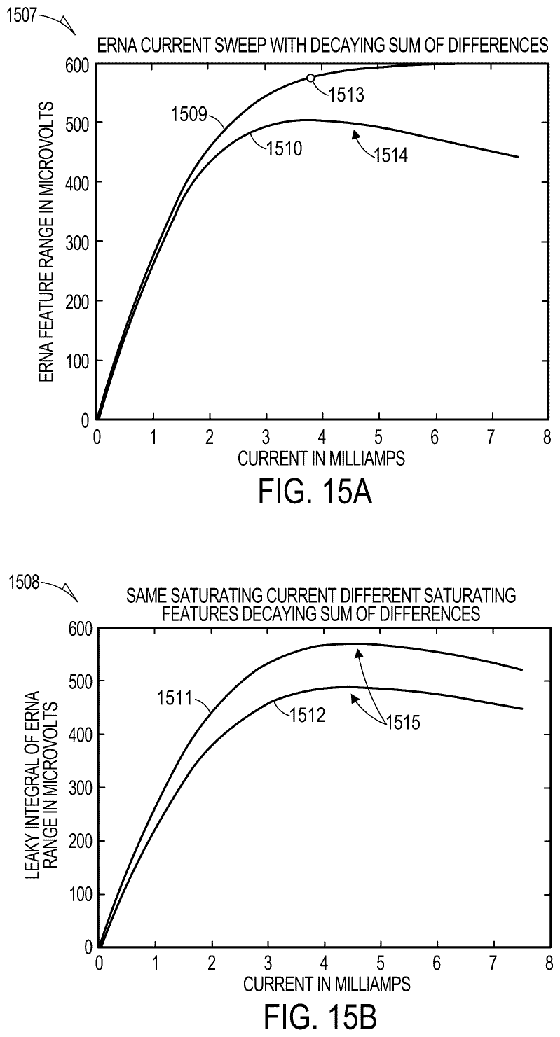

FIGS. 14A-14B illustrate, by way of example and not limitation, saturation distributions for detecting saturation. FIG. 14A indicates an example where a saturation value is the same value for ERNA feature and the sum of differences but the saturation value current is different. For example, the decaying sum of differences 1403 and the recorded ERNA feature 1404 plateaus or levels out at a same amplitude (shown on the y-axis) while the current (shown on the x-axis) at which it saturates is different. In the reverse, in one example, FIG. 14B indicates an example where a saturation value is different for the ERNA feature and the sum of differences but the saturation value current is the same. For example, the decaying sum of differences 1406 and the recorded ERNA feature 1405 plateaus or levels out at a different amplitude (shown on the y-axis) while the current (shown on the x-axis) at which it saturates is the same.

FIGS. 15A-15C illustrate, by way of example and not limitation, saturation distributions including maxima for detecting saturation. In FIG. 15A, the saturation distributions include an ERNA feature distribution 1509 and a sum of decaying differences 1510. As referenced at 1395 in FIG. 13, the decay rate of the sum of differences of consecutive recordings is below a particular threshold at point 1514 and therefore the corresponding saturation point of the ERNA feature is at point 1513. As an example, the differences in consecutive recordings decreases to a particular low threshold value such that the saturation point has been approached or achieved.

In FIG. 15B, the maxima of saturating current occurs at a same current value even with different feature amplitudes. As an example, the distribution 1511 illustrates a decaying sum of differences when a saturating current is small and the distribution 1512 illustrates a decaying sum of differences when a saturating current is large. The x-axis corresponds to a leaky integral of the ERNA range in microvolts and the y-axis corresponds to a current (in millamps). As shown, the maximas 1515 occur at a same current value.

In contrast, in FIG. 15C, the maxima of saturating current occurs at a different current value with different feature amplitudes. As an example, the distribution 1516 illustrates a decaying sum of differences when a saturating current is small and the distribution 1517 illustrates a decaying sum of differences when a saturating current is large. The x-axis corresponds to a leaky integral of the ERNA range in microvolts and the y-axis corresponds to a current (in millamps). As shown, the maximum 1518 of distribution 1516 is at a lower current than the maximum 1519 of the distribution 1517.

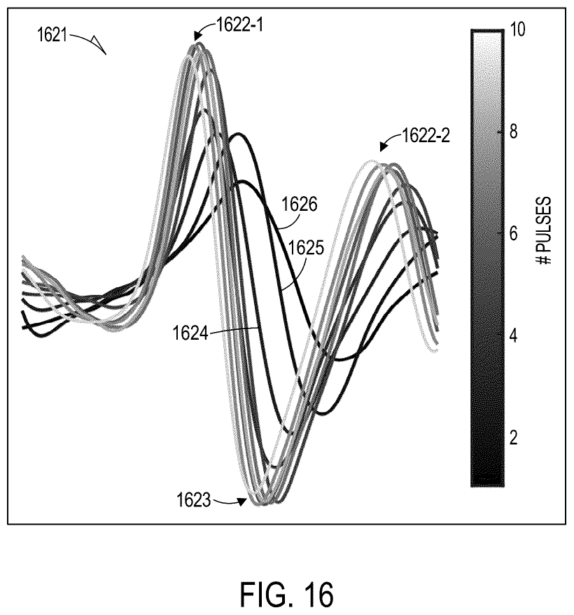

FIG. 16 illustrates, by way of example and not limitation, distributions of ER dynamics at a particular DBS intensity. In this example, an intensity of 2.5 milliamps is used for purposes of illustration. The distributions 1621 each have a first peak 1622-1 and a second peak 1622-2 and a first trough 1623. As is illustrated, a first distribution 1626 (corresponding to approximately one pulse) has a lower first peak and second peak and a higher trough than the other distributions. A second distribution 1625 (corresponding to approximately two pulses) has a higher first peak and second peak than the first distribution 1626 and a lower first trough than the first distribution 1626. Likewise, a third distribution 1624 has higher first and second peaks and a lower first trough than the first and second distributions 1626, 1625. In this way, FIG. 16 illustrates how changing dynamics of different number of pulses has an affect on ERNA behavior and can be used as an ERNA feature to distinguish particular parameters of the patient. For example, these particular dynamics of the ERNA signal can be used to determine a state or condition of the patient and to adjust or modify the neuromodulation treatment parameters in order to provide optimal or more effective therapy to the patient.

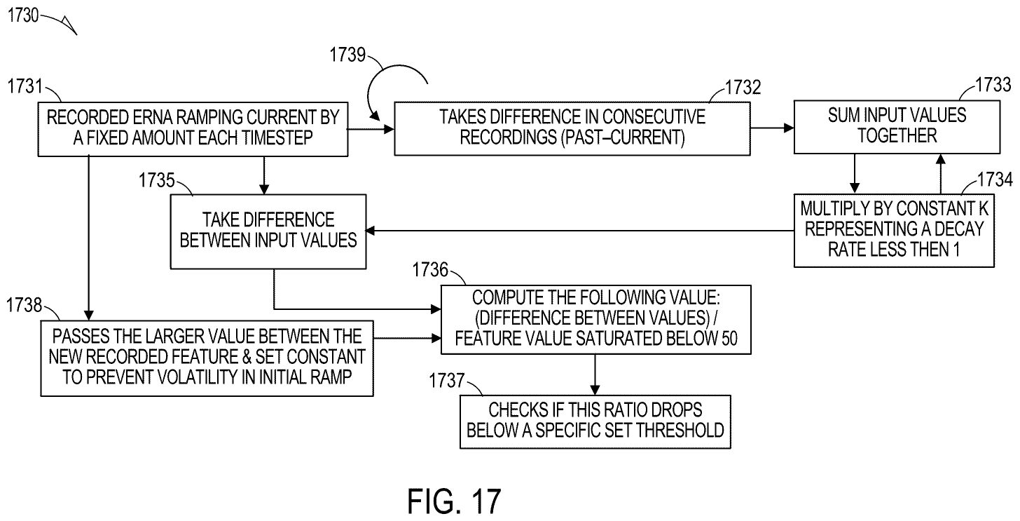

FIG. 17 illustrates, by way of example and not limitation, an example of a method for saturation detection. The method 1730, as an expansion of the method 1390 in FIG. 13, can include recording ERNA signals while increasing a current by a fixed amount. The current can be increased at particular time intervals. The particular time intervals can be based on previous information that indicates which time intervals are correlated with a particular increase or change in ERNA features.

At 1732, a difference in consecutive ERNA signals can be determined. In some examples, prior ERNA signal recordings can be subtracted from current ERNA signal recordings. At 1733, the input values of the differences can be summed together. For example, the sum of the differences can be illustrated along a continuum over time, as in FIGS. 14A-15C. At 1734, the summed input values can be multiplied by a constant K, where the constant K represents a decay rate that is less than 1. By multiplying the summed input values by the constant K, a decay rate factor can be introduced into the summed input values. The constant K can be based on a prior model or previous data used to determine a decay rate.

At 1735, the difference between the input values, e.g., a difference between the feature values and the decayed summed input values, can be provided as an input. At 1736, compute the following ratio value: (Difference Between Values)/Feature Value saturated below 50. Dividing by the Feature Value saturated below 50 prevented a large ratio value for small recorded ERNA amplitudes (for example if the amplitude was 0.001, the resulting ratio would be very large).

At 1738, passes the larger value between the new recorded feature and the set constant to prevent volatility in the initial ramping up. This refers to tracking a maximum recorded value and passing along the maximum value up to that point along to the next step. As an example, a first value of 400 microvolts can be received. The value of 400 microvolts would be passed along. A subsequent second value of 200 microvolts can be received. The value of 400 microvolts would be passed along again as it is greater than the recently received 200 microvolts. A third value of 600 microvolts can be received. The value of 500 microvolts would then be passed along as it is greater than 400 microvolts and 200 microvolts. At 1737, a determination of whether the ratio value is below a specific set threshold can be performed.

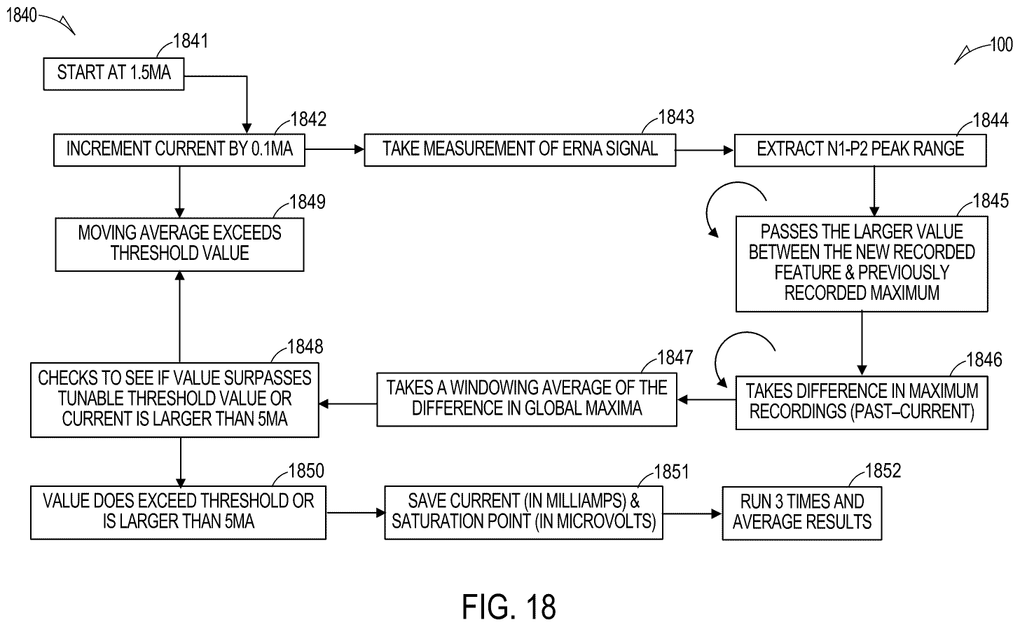

FIG. 18 illustrates, by way of example and not limitation, an example of a method for saturation detection. The method 1840 can begin, at 1841, by using an amplitude of 1.5 milliAmps (mA). While 1.5 mA is used in this example, examples are not so limited and any number of currents can be used to begin. At 1842, the current is incremented by 0.1 mA for each cycle. At 1843, a measurement of the ERNA signal is recorded. At 1844, an N1-P2 peak range is extracted from the recorded data. An N1-P2 peak range refers to the difference between a first trough (N1) and a second peak (P2). For example, as illustrated in FIG. 12B, the N1-P2 peak range is a range from a first trough 1287 (which is N1) to a second peak 1285 (which is P2).

At 1845, the larger value is passed between the new recorded feature and the previously recorded maximum. At 1846, the difference in maximum recordings are determined. For example, a difference between a prior maximum and a current maximum are determined. At 1847, a windowing average of the difference in global maxima is determined. At 1848, a determination of whether a value surpasses an adjustable threshold value or whether the current is larger than 5 mA is performed. At 1849, in response to the moving average exceeding the adjustable threshold value, the current can be incremented by 0.1 mA. At 1850, a determination of whether the value does not exceed the adjustable threshold value or whether current is larger than 5 mA can be performed. At 1851, the current (in milliamps) and saturation point (in microvolts) can be saved. At 1852, the method can be performed at least three times and the results can be averaged.

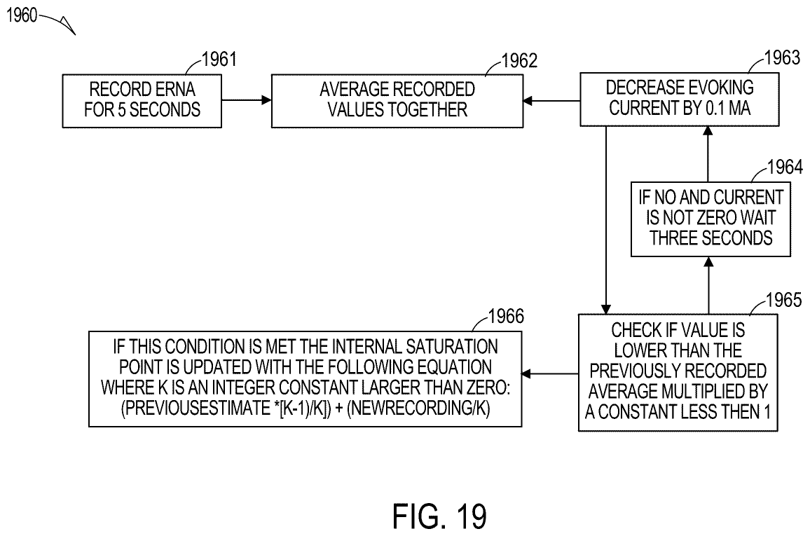

FIG. 19 illustrates, by way of example and not limitation, an example of a method for recalibration a saturation point. The method 1960 can include, at 1961, recording ERNA signals for at least 5 seconds. At 1962, the recorded values from the ERNA signals can be averaged. At 1963, an evoking current can be decreased by 0.1 mA. At 1965, a determination whether the value is lower than a previously recorded average multiplied by a constant less than 1 can be performed. At 1964, in response to the value not being lower than the previously recorded average multiplied by the constant, and in response to the current not being zero, a delay of 3 seconds can be introduced. At 1966, in response to the value being lower than the previously recorded average multiplied by the constant, an internal saturation point is updated with the following where K is an integer constant larger than zero: (PreviousEstimate*[(K−1)/K])+(NewRecording/K).

As used herein, the term “machine readable medium” may include any medium that is capable of storing, encoding, or carrying instructions for execution by a processing device or machine and that causes the processing device or machine to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
delivering electrostimulation to a neural target of a patient in accordance with a stimulation setting via a stimulating electrode selected from a plurality of electrodes on at least one lead;
sensing evoked responses (ERs) using sensing electrodes connected to a sensing circuit, the sensing electrodes selected from the plurality of electrodes on the at least one lead;
using a processing system to:
determine a first stimulation threshold for a stimulation parameter based on a first sensed threshold of the sensed ERs, wherein the first stimulation threshold indicates a minimum value or a maximum value for the first sensed threshold;
determine a second stimulation threshold for the stimulation parameter based on additional therapy parameters, wherein the second stimulation threshold indicates a maximum value for the sensed ERs; and
deliver electrostimulation therapy to the neural target using a stimulation parameter set to cause a subsequent sensed ER value to be above the minimum value or below the maximum value and below the second stimulation threshold.

2. The method of claim 1, further comprising using the processing system to detect a saturation point associated with the stimulation parameter;
wherein the saturation point is detected based on a comparison of a difference in consecutive electrostimulation recordings.

3. The method of claim 2, wherein the difference in consecutive electrostimulation recordings comprises a comparison of a first-ordered trough of a stimulation wave to a second-ordered positive peak of the stimulation wave.

4. The method of claim 1, wherein:
the electrostimulation therapy is delivered based on additional sensed parameters; and
the additional sensed parameters comprise a frequency of at least one of the sensed ERs, an N1-P2 peak amplitude, an N1-P2 peak delay, a size of an evoked potential envelope, or a relative amplitude of each peak.

5. The method of claim 4, further comprising using the processing system to use at least one of the additional sensed parameters to determine a state of the patient, wherein the state of the patient comprises one of a sleep state, an awake state, a state of receiving medication, or a state of diminishing medication release, sufficient therapy, insufficient therapy, or an experience of at least one side effect.

6. The method of claim 1, further comprising using the processing system to:
update the first stimulation threshold based on a change of state of the patient; or
update the first stimulation threshold second stimulation threshold based on a change of state of the patient.

7. The method of claim 1, further using the processing system to:
recalibrate the first stimulation threshold and the second stimulation threshold; and
adjust the stimulation parameter to cause the subsequent sensed ER value to be above a minimum value or a maximum value each associated with the recalibrated first stimulation threshold or below the recalibrated second stimulation threshold.

8. The method of claim 1, further comprising using the processing system to change the stimulation parameter associated with the delivered electrostimulation in response to the stimulation parameter causing the sensed ERs to be below the first threshold or above the second threshold.

9. The method of claim 1, further comprising using the processing system to determine a state of the patient and adjusting the delivered electrostimulation to a predetermined stimulation value associated with the determined state.

10. The method of claim 1, wherein the sensing electrodes include two or more electrodes immediately adjacent to the stimulating electrode on the at least one lead.

11. The method of claim 1, wherein the at least one lead includes a deep brain stimulation (DBS) lead, and wherein the electrostimulator is configured to provide DBS to a brain target of the patient.

12. The method of claim 1, further comprising using the processing system to determine the first stimulation threshold or the second stimulation threshold based on a particular time period that the electrostimulation is delivered.

13. The method of claim 1, further comprising using the processing system to determine the first stimulation threshold or the second stimulation threshold based on a particular activity being performed by the patient.

14. The method of claim 1, further comprising using the processing system to provide stimulation values for the stimulation parameter set based on at least one of a time of day that the electrostimulation is performed and a particular activity being performed by the patient.

15. A non-transitory computer-readable medium storing instructions executable by a processor to:
deliver electrostimulation to a neural target of a patient in accordance with a stimulation setting via a stimulating electrode selected from a plurality of electrodes on at least one lead, wherein the electrostimulation is delivered to the neural target of the patient via a medical-device system that comprises an electrostimulator and the at least one lead coupled thereto;
sense evoked responses (ERs) using sensing electrodes connected to a sensing circuit, the sensing electrodes selected from the plurality of electrodes on the at least one lead;
determine a first stimulation threshold for a stimulation parameter based on a first sensed threshold of the sensed ERs, wherein the first stimulation threshold is a stimulation value at which a change in the sensed ERs occurs;
determine a second stimulation threshold for the stimulation parameter based on additional therapy parameters, wherein the second stimulation threshold indicates a maximum value for the stimulation parameter; and
deliver electrostimulation therapy to the neural target using a stimulation parameter set to cause a subsequent sensed ER value to be above or below the first stimulation threshold and below the second stimulation threshold.

16. The non-transitory computer-readable medium of claim 15, wherein the instructions are executable by the processor to deliver the electrostimulation therapy based on additional sensed parameters, wherein the additional sensed parameters comprise a frequency of at least one of the sensed ERs, an N1-P2 peak amplitude, an N1-P2 peak delay, a size of an evoked potential envelope, or a relative amplitude of each peak.

17. The non-transitory computer-readable medium of claim 16, wherein the instructions are executable by the processor to use at least one of the additional sensed parameters to determine a state of the patient, wherein the state of the patient comprises one of a sleep state, an awake state, a state of receiving medication, or a state of diminishing medication release, sufficient therapy, insufficient therapy, or an experience of at least one side effect.

18. The non-transitory computer-readable medium of claim 15, wherein the instructions are executable by the processor to update the first stimulation threshold based on a change of state of the patient or update the second stimulation threshold based on a change of state of the patient.

19. The non-transitory computer-readable medium of claim 15, wherein the instructions are executable by the processor to change at least one of a plurality of stimulation parameters associated with the delivered electrostimulation in response to the stimulation parameters causing the sensed ERs to be below or above the first sensed threshold or above the second sensed threshold.

20. A system, comprising:
at least one lead including a plurality of electrodes;
an electrostimulator configured to provide electrostimulation to a neural target of a patient;
a sensing circuit configured to sense an evoked response (ER) to the electrostimulation; and
a controller circuit operably connected to the electrostimulator and the sensing circuit, the controller circuit configured to:
deliver the electrostimulation to the neural target in accordance with a stimulation setting via at least one stimulating electrode selected from the plurality of electrodes on the at least one lead;
collect sensed ERs to the electrostimulation using at least one sensing electrode on the at least one lead; and
determine a first stimulation threshold for a stimulation parameter based on a first sensed threshold of the collected sensed ERs, wherein the first stimulation threshold is a stimulation value at which a change in the collected sensed ERs occurs.
